(19) United States
(12) Patent Application Publication
(10) Pub. No.: US 2008/0033322 A1
(43) Pub. Date: Feb. 7, 2008

(54) Systems and methods for measuring cervical dilation

(75) Inventors: Gerald Feuer, Atlanta, GA (US); Gerald J. Sanders, Sonoma, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSUITE, LLC, San Francisco, CA (US)

(21) Appl. No.: 11/496,884

(22) Filed: Jul. 31, 2006

(51) Int. Cl. A61B 5/103

(52) U.S. Cl. 600/588

(57) ABSTRACT

Systems and methods for measuring cervical dilation are provided that include two biocompatible fasteners affixed to opposing cervical or vaginal walls, and a biocompatible string coupled to the fasteners, yet free to slide between the fasteners. The string has ends extending outside the vaginal opening of the patient, so that changes in cervical dilation can be measured by monitoring the relative movements of the string ends. The heads of the screws are preferably concave, with a loop attached thereon, so that the string can be coupled to the screws by running the string therethrough. Further, the tips of the screws are preferably blunt, to minimize loss of blood and the possible risk of HIV infections.

### FIELD OF THE INVENTION

The present invention relates to the measurement of cervical dilation during childbirth. More particularly, the present invention relates to systems and methods for measuring cervical dilation that include affixing biocompatible screws to opposing cervical walls, slidably connecting a biocompatible string to the screws, and measuring the retraction of the string as the cervix dilates.

### BACKGROUND OF THE INVENTION

Cervical dilation is a critical indicator of the effectiveness of uterine contractions and of the progress of labor during the childbirth process. By measuring cervical dilation, a clinician can not only monitor the advancement of the fetus, but can also determine the most appropriate time for a patient to begin pushing, or when to administer labor stimulants or anesthetic agents. The extent of cervical dilation is also an important indicator for diagnosing abnormal conditions such as cephalopelvic disproportion or dysfunctional labor, and to decide whether a cesarian section has become necessary.

Cervical dilation measurements are typically performed through digital (finger-based) examinations, during which a clinician inserts the first and second fingers of a gloved hand into the vaginal canal and spreads them into the cervix. Cervical dilation is then empirically measured through an assessment of the distance between the two fingers. Therefore, the quality of this empirical measurement system is heavily dependent on the experience of the clinician and is inherently prone to error.

Because the clinician's digital measurements are typically compared against reference labor curves (referred to as the “Friedman curves” gin the medical profession) that plot cervical dilation vs. time, a clinician's ability to determine whether a patient's labor is normal or dysfunctional, and to act accordingly in a timely fashion, is severely limited by the inherent unreliability of the data points used to build the patient's labor curves.

Despite the use of gloves, digital vaginal exams may also cause infections of the fetal membranes (chorioamnionitis), of the lining and muscle of the uterus (endomyometritis), or of the infant (neonatal sepsis). Infection risks increase dramatically after the fetal membranes have been ruptured, forcing the clinician to limit the number of vaginal exams after the rupturing of the membranes protecting the amniotic cavity.

Devices have been proposed in the prior art to measure cervical dilation. For example, U.S. Pat. No. 4,141,345 to Allen et al. discloses a device for measuring cervical dilation that is essentially a V-shaped caliper having ends attached to opposite cervical walls. While providing a more reliable measurement than digital examination, Allen's device creates a physical barrier to the passage of the fetus, and is not disposable, requiring repeated sterilizations.

U.S. Pat. No. 5,658,295 to Krementsov discloses a scissors-like device having tips in contact with the cervical walls at one end, and a measuring gauge at the other end. Krementsov's device also creates a physical barrier to the passage of the fetus and is not disposable, requiring repeated sterilizations.

U.S. Pat. No. 6,039,701 to Sliwa et al. discloses a support structure that detects changes in cervical diameter by detecting changes in the size of the support structure. In its different embodiments, Sliwa's invention involves devices that require a plurality of components, that still create physical barriers to the passage of the fetus, and that are also not disposable, requiring repeated sterilizations.

U.S. Pat. No. 6,966,881 to Ben-Cnaan et al. discloses a cervical dilation monitor that includes an expandable device having opposing members that grip the cervical walls. Ben-Cnaan's device still creates a physical barrier to the passage of the fetus and is not disposable.

Therefore, it would be desirable to provide systems and methods for measuring cervical dilation that generate reliable and continuous measurements of the spreading of the cervical walls.

It would also be desirable to provide systems and methods for measuring cervical dilation that do not interfere with the passage of the fetus during childbirth.

It would further be desirable to provide systems and methods for measuring cervical dilation that are inexpensive to manufacture and disposable after use.

### SUMMARY OF THE INVENTION

In view of the foregoing, it is an object of the present invention to provide systems and methods for measuring cervical dilation that overcome the drawbacks of previously-known designs and that provide a simple yet reliable indications of labor progress.

It is another object of the present invention to provide systems and methods for measuring cervical dilation that enable a clinician to assess changes in the cervical opening on a continuous basis.

It is also an object of the present invention to provide systems and methods for measuring cervical dilation that create no obstacles to the passage of the fetus through the vaginal canal upon childbirth.

It is yet another object of the present invention to provide systems and methods for measuring cervical dilation that are fast and simple to implement by a clinician.

It is a further object of the present invention to provide systems and methods for measuring cervical dilation that are inexpensive to manufacture and that are disposable after use.

These and other objects of the present invention are accomplished by providing systems and methods for measuring cervical dilation that include two biocompatible fasteners affixed to opposing cervical or vaginal walls, and a biocompatible string that is connected to the fasteners, yet that remains free to slide. The string has ends disposed outside the vaginal opening of the patient, so that changes in cervical dilation can be measured by measuring length changes in that portion of the string that extends outside the vaginal opening.

In one embodiment, the fasteners are screws that have shafts less than 5 mm long, to prevent a complete perforation through the cervical wall and contact with the fetus, both while the screws are being attached and later during childbirth. The heads of the screws are preferably concave, with a loop attached thereon, so that the string can be connected to the screws by running the string through the loops. Additionally, the tips of the screws are blunted, to minimize the risk of loss of blood and the related risk of HIV infection.

The screws may be made of a bio-absorbable material, for example, a polyglactic material, so that the screws can be left in place after childbirth and be absorbed by the patient's body over time.

In other embodiments, relative movements of the string ends are measured with a measuring device, and bell shaped clips may be positioned between the screws and the cervical walls, retaining any blood lost during screw penetration and acting as tamponades against the screw wounds.

An applicator may also be provided that enables a clinician to affix the screws to the cervical walls while operating from outside the vaginal opening. This applicator has a tubular shape with a lumen therethrough, and a push rod that reciprocates within the lumen and that extends from the proximal end of the applicator. The distal end of the applicator is preferably beveled, to enable the clinician to have adequate contact with the cervical walls without tilting the applicator. When the clinician applies pressure on the push rod, a screw inside the lumen is pushed into the cervical wall, becoming affixed to the cervical wall.

In one embodiment, the applicator has two lumens and two push rods. This double-lumen construction enables the clinician to insert a fastener on each of the opposing cervical walls without removing the applicator from the vaginal canal.

Methods for measuring cervical dilation are also disclosed.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a schematic view of the system of the present invention used in a patient;

FIGS. 2A-2B are side views of two different screws according to two different embodiments of the present invention;

FIG. 3 is a side view of a screw with retaining clip according to another embodiment of the present invention;

FIG. 4 is a side view of an applicator suitable for inserting screws in a patient's cervical walls; and

FIG. 5 is a detail view of the distal end of the rod in the applicator of FIG. 4.

### DETAILED DESCRIPTION OF THE INVENTION

The present invention is directed to systems and methods for measuring cervical dilation that include the insertion of fasteners into opposite cervical or vaginal walls, and the measurement of the distance between the fasteners during dilation. In a preferred embodiment, the fasteners are screws with concave heads, and a string is slidably connected to the screws. The ends of the string are disposed outside of the vaginal opening, and cervical dilation is measured by measuring changes in that portion of the string that extends outside of the vaginal opening.

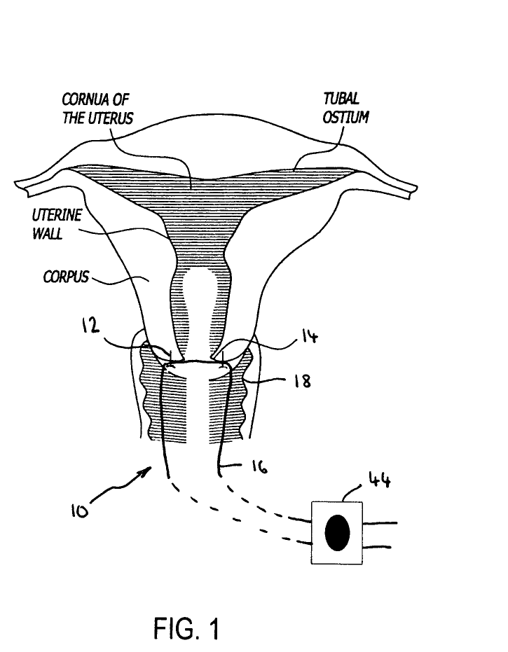

Referring first to FIG. 1, an exemplary embodiment of a system for measuring cervical dilation in accordance with the principles of the present invention is described. System 10 includes two screws 12, which are inserted into opposite walls 14 of the cervical os, and a string 16, which is connected to both screws 12 yet is free to slide between screws 12. Although the present description relates to an embodiment in which screws 12 are inserted into opposite walls 14 of the cervical os, in a different embodiment screws 12 may be inserted into opposing vaginal walls 18 rather than on opposing os walls 14. The ends of string 16 are situated outside of the vaginal opening, so that a portion of the string extends outside the vaginal opening. If string 16 is formed as a closed loop, the portion of the string loop opposite to the cervix is the portion of string 16 that extends out of the vaginal opening.

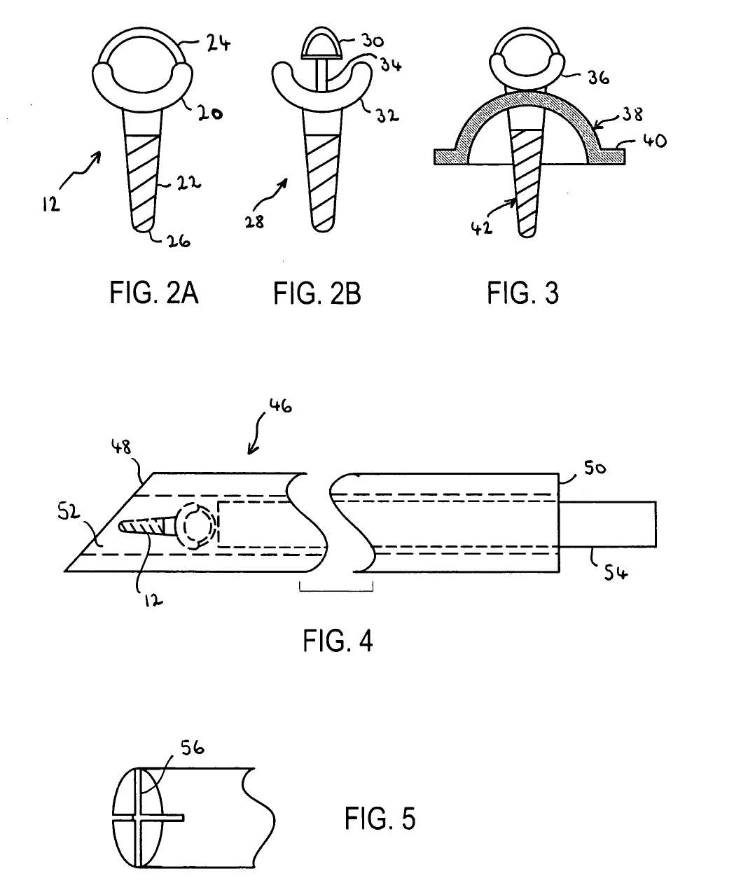

Referring now to FIGS. 2A-2B, screw 12 is described in greater detail. Screw 12 includes a head 20 and a shaft 22, which is threaded. Because screws 12 may be inserted into opposite walls 14 of the cervical os, and because the thickness of os walls 14 decreases as the cervix becomes dilated, shaft 22 is preferably no longer than 5 mm for a normal patient, typically 3-4 mm long. Generally, screws 12 are inserted in the early stages of dilation, for example, when dilation reaches 3 cm.

Head 20 is preferably concave, to achieve a more pronounced insertion into os walls 14 or vaginal walls 18. When screws 12 are inserted into os walls 14, the concave construction of head 20 also minimizes contact with the vaginal tunic when the cervix becomes fully dilated during childbirth and the cervical wall is pushed out, contacting laterally the vaginal tunic. Further, the concave contour of head 20, as opposed to a convex contour, minimizes discomfort to the patient if screws 12 are left inserted in os walls 14 after childbirth.

In one embodiment, string 16 is anchored to screw 12 by rolling string 12 around shaft 22 under head 20. In another embodiment, depicted in FIG. 2A, a loop 24 extends from head 20 to connect string 16 with screw 12, allowing string 16 to be coupled to screw 12 by being inserted through loop 24, remaining free to slide through loop 24 when cervical wall 14 increasingly dilates as childbirth progresses. While loop 24 is depicted in FIG. 2A as a convex circular segment, one skilled in the art will recognize that other contours of loop 24 are within the scope of the present invention. To mention just one example, loop 24 may be shaped with a concave contour nested within the concavity of head 20.

In still another embodiment, depicted in FIG. 2B, loop 30 is connected to head 32 through stem 34 that may be free to rotate, so that string 16 may be inserted into loop 34 prior to the insertion of screw 28 into cervical wall 14, and will not twist on itself while screw 28 is being inserted.

Al least a portion of shaft 22 is threaded, to provide for an optimal anchoring to cervical walls 14 or vaginal walls 18. Additionally, tip 26 of shaft 22 is blunted, for example, with the same blunted contour as a liver suture needle, in order to minimize the risk of HIV infection if blood is released from the screw wound in cervical wall 14.

Screw 12 and string 16 are manufactured from a bio-compatible material, that may be non-absorbable, for example nylon, polyester, or polypropylene, or bio-aborbable, for example polyglycolic acid, lactic acid, or caprolactone. One bio-absorbable material is polyglactin, which is commercially available, among others, under the trade name VICRYL (which relates to polyglactin 910), and which is widely employed in the manufacture of surgical sutures. When VICRYL is employed, screws 12 may be left anchored in cervical walls 14 after childbirth and become absorbed by the patient's body over time.

String 16 may have open ends, or ends attached one to the other, making string 12 a closed loop. Both ends of string 16 are disposed outside of the vaginal opening, and when cervical walls 14 dilate, screws 12 become increasingly spaced apart from each other, pulling a larger portion of string 16 into the vaginal canal. Consequently, the ends of string 16 retract in the direction of the vaginal opening, and the retracting action of the cervical walls enables a clinician to measure the progress of cervical dilation by measuring the amount of string 16 that is pulled into the vaginal opening over time.

In one embodiment, string 16 carries gauge marks along its length, to provide the clinician with a measure of cervical dilation by counting the number of gauge marks remaining outside the vaginal opening and by comparing that count with an earlier count. In another embodiment, the gauge marks have different colors, so that the clinician can make an immediate assessment of the amount of cervical dilation by observing which gauge mark color has become nearest to the vaginal opening in comparison to a previous observation. In still another embodiment, one end of string 16 is maintained in a constant position, and cervical dilation is measured by observing the movement of the other end of string 16. For example, one end of screw 16 may be attached to a belt wrapped around the patient's waist or to a leg of the patient, and the other end may be kept loose; or string 16 may have a fixed end and unwind from a reel. The measurement of the movements of one or both ends of string 16 may also be facilitated by having opposing sides of string 16 move within a slotted fitting, commonly identified as “Texas tie” gin the mechanical trade, or by measuring the relative movement of opposing string sides through a measuring device.

Referring now to FIG. 3, a different mode of screw attachment to cervical walls 14 is shown. Screw 36 is supported by a clip 38, which is bell shaped and which includes a flange 40 extending around its base to increase contact surface with cervical wall 14. The main purpose of clip 38 is to create a tamponade-like effect after insertion of screw 42 into cervical wall 14, in the event that blood is released by the screw wound. Clip 38 contains the blood, increasing the speed of coagulation of the blood and the consequent healing of the wound. Clip 38 also minimizes fetus and clinician contact with the patient's blood, minimizing the risk of HIV or other infections. One skilled in the art will recognize that “bell shape” is indicative not of one specific shape of clip 28 but of a variety of shapes, whether semi-spherical, elongated, or otherwise, all within the scope of the present invention. One skilled in the art will also recognize that the length and threading of screw 42 must be adjusted in relation to the shape and size of clip 38.

Referring now to FIGS. 4-5, an applicator 46 that enables the affixing of screws 12 to cervical walls 14 while operating from outside the vaginal opening is described. In one embodiment, applicator 46 comprises a distal end 48, a proximal end 50, and a lumen 52 extending between distal end 48 and proximal end 50. Distal end 48 preferably has a beveled edge, to facilitate contact with cervical wall 14 without requiring the clinician to tilt applicator 46 within the vaginal canal. A rod 54 reciprocates within lumen 52 and extends outside of proximal end 50, so that, when a screw 12 is inserted in lumen 52 and rod 54 is pressed towards distal end 48, screw 12 is pushed out of lumen 42 and into cervical wall 14.

Rod 54 may have a flat distal end, applying pure compressive pressure on screw 12, or may have a distal end carrying grooves 56, within which loop 24 may rest, so that screw 12 may be affixed to cervical wall 14 with a twisting motion. Alternatively, screw 12 may be affixed to cervical wall 14 using a screwdriver-type device that has a distal end embossed with grooves 56.

String 16 may be inserted into loop 24 prior to the application of screw 12 to cervical wall 14. If screw 12 is affixed to cervical wall 14 with a clockwise motion, string 16 may be pre-twisted around loop 24 in a counter-clockwise direction, so that no twist essentially remains on string 16 after screw 12 is affixed to cervical wall 14.

In a different embodiment, applicator 46 may be configured with two parallel lumens 52 and two parallel rods 54 that provide for the insertion of two screws 12. With this embodiment, the clinician can affix both screws to cervical walls 14 without removing the applicator from the vaginal canal.

A method for measuring cervical dilation according to the principles of the present invention is now described. In a first step, the clinician inserts two screws 12 into opposing cervical walls 14. In applying screws 12, the clinician may be assisted by the use of applicator 46; preferably, the clinician will make use of a double lumen applicator, so that both screws 12 can be affixed to cervical wall 14 without removing applicator 32 from the vaginal canal.

In a second step of the present method, string 16 is slidably connected to both screws 12. Alternatively, string 16 may be connected to screws 12 prior to affixing screws 12 to cervical wall 14. When screw 12 is provided with loops 24, string 16 is inserted through loops 24, so that string 12 becomes coupled with screws 12 but still retains the ability to slide within loops 24 as the cervix dilates and cervical walls 14 move further apart.

The ends of string 16 extend outside of the vaginal opening, and the amount of string 16 that is retracted inside the vaginal opening is monitored by a clinician. In an alternative embodiment, string 16 has no free ends but is configured like a closed loop, in which case it is the side of the closed loop opposite to the cervix that extends outside of the vaginal opening and that is monitored by a clinician.

In a third step, the clinician measures the dilation of the cervix by measuring the amount of string 16 that has been pulled inside the vaginal opening due to the separation of cervical walls 14. The clinician's measurement may be aided by observing the movement of gauge marks disposed on string 16, for instance, by counting how many gauge marks have been pulled into the vaginal canal or how many gauge marks are left on the portion of the string outside the vaginal canal. If the gauge marks have a plurality of colors, the clinician may simply observe which color is nearest to the vaginal opening, and relate that color to the color that was closest to the vaginal opening at the beginning of the measuring process. Alternatively, the clinician may make use of a measuring device.

Upon the full opening of the cervix, for example, during childbirth, cervical walls 14 become pushed outwards and contact laterally the walls of the vaginal tunic. During that process, loops 24 and screw heads 20 become enclosed between cervical wall 14 and the vaginal tunic, protecting the fetus from contact with screws 12. At the same time, string 16 provides no impediment to the movement of the fetus as it can be easily pushed out by the fetus during childbirth, or can be slid off and removed from loops 24.

After childbirth, screws 12 may be removed or, if screws 12 are manufactured fro a bio-absorbable material, may be left implanted in cervical walls 14, progressively dissolving over time.

In a different embodiment of the invention, a method for measuring cervical dilation includes the use of two screws 12 but not the use of a string. Screws 12 are inserted into cervical walls 14 in the same manner as in the above described method, but their relative position is monitored instead with the aid of appropriate equipment, for example, with a sonography machine that determines screw position. As the cervix becomes dilated, the screws become positioned further apart, and the sonography machine provides the clinician with a determination of screw positions, and therefore of cervical dilation.

In a variant of this embodiment, one of screws 12 may contain metal, while the tip of the other may operate as a soundwave transmitter, signaling the relative position of screws 12 to a machine. In another variant of this embodiment, both screws 12 contain metal, and their relative position is detected with the aid of a metal-detecting machine.

While preferred embodiments of the invention are described above, it will be apparent to one skilled in the art that various changes and modifications may be made. The appended claims are intended to cover all such changes and modifications that fall within the true spirit and scope of the invention.

1. A system for measuring cervical dilation in a patient, the system comprising:
two bio-compatible screws adapted to be affixed to opposing cervical or vaginal walls; and
a bio-compatible string slidably coupled to the screws, the string having a first portion adapted to be contained within the patient's vaginal opening and a second portion adapted to be extending outside the vaginal opening, the string further having ends adapted to be disposed outside of the patient's vaginal opening,
wherein changes in the cervical dilation are measured by measuring changes in the length of the second portion of the string;
an applicator configured to allow affixing of the screws from outside the vaginal opening.

2. (canceled)

3. The system of claim 1, wherein the screws have a shaft less than 5 mm long.

4. The system of claim 1, wherein the screws have concave heads each with a loop attached thereon, and wherein the string is coupled to the screws by inserting the string through the loops.

5. The system of claim 2, wherein the screws have blunt tips.

6. The system of claim 1, wherein the screws are made of a bio-absorbable material.

7. The system of claim 6, wherein the screws are made of a polyglactic material.

8. The system of claim 1, wherein the string is made of a polyglactic material.

9. The system of claim 1, wherein one of the string ends is connected to a fixed reference point, and wherein the other string end is free to slide.

10. The system of claim 9, wherein the fixed reference point is situated on a belt surrounding a portion of a body of the patient.

11. The system of claim 1, wherein the ends of the string are slidably retained within a slotted fitting.

12. The system of claim 1, wherein the changes in the length of the second portion of the string are measured with a measuring device.

13. The system of claim 1, wherein the string ends are connected one to the other.

14. The system of claim 1, further comprising a plurality of bell shaped clips disposed around the screws and adapted to be disposed against the cervical or vaginal walls, the bell shaped clips acting as tamponades.

15. (canceled)

16. The system of claim 1, wherein the applicator has an elongated shape with a distal end, a proximal end, and a lumen therebetween, wherein a push rod reciprocated within the lumen and extends from the proximal end, and wherein pressure on the push rod causes a screw, of the two screws, situated in the lumen to be inserted in one of the cervical or vaginal walls.

17. The system of claim 16, wherein the applicator has two lumens and two push rods, thereby enabling the clinician to insert a screw, of the two screws, on each of the opposing cervical or vaginal walls without removing the applicator from the vaginal canal.

18. The system of claim 16, wherein the distal end has a beveled profile.

19. A method for measuring cervical dilation in a patient, the method comprising:
affixing two bio-compatible screws to opposing cervical or vaginal walls;
slidably coupling a bio-compatible string to the two screws;
extending the opposing ends of the string out of the patient's vaginal opening, causing the string to have a first portion contained within the patient's vaginal opening and a second portion extending outside the vaginal opening; and
measuring changes in the cervical dilation by measuring changes in the length of the second portion of the string;
wherein the screws are adapted to be affixed to the cervical or vaginal walls by using an applicator.

20. The method of claim 19, wherein the screws have concave heads each with a loop attached thereon, the screws further having rounded tips and shafts less than 5 mm long, and wherein the string is coupled to the screws by inserting the string into the loops.

21. The method of claim 19, wherein the screws are made of a bio-absorbable material, wherein the screws remain affixed to the cervical or vaginal walls after cervical dilation is measured, and wherein the screws are absorbed by the patient's body over time.

22. The method of claim 21, wherein changes in the length of the second portion of the string are measured by attaching one end of the string to a fixed point and measuring the displacement of the opposing end.

23. The method of claim 19, wherein changes in the length of the second portion of the string are measured with the aid of a measuring device.

24. The method of claim 19, further comprising the steps of applying wherein each screw of the two screws further includes a bell shaped clip configured to be disposed around each screw and against the cervical or vaginal walls and further configured to create a tamponades-like effect after insertion of the screws into the cervical or vaginal walls.

25. The method of claim 19, wherein the applicator has an elongated shape with a distal end, a proximal end, and a lumen therebetween,
wherein a push rod reciprocates within the lumen and extends from the proximal end, the push rod being maneuverable outside of the vaginal opening by a clinician,
wherein the distal end is positioned against one of the cervical or vaginal walls, and wherein pressure by the clinician on the push rod causes a screw of the two screws situated in the lumen to become affixed to one of the cervical or vaginal walls.

26. The method of claim 25, wherein the applicator has two lumens, thereby enabling the clinician to insert a screw of the two screws on each of the opposing cervical or vaginal walls without removing the applicator from the vaginal canal.

27. A method for measuring cervical dilation, the method comprising:
affixing a plurality of biocompatible screws to opposing cervical or vaginal walls during cervical dilation;
wherein the movement of the screws is detectable by a non-invasive, machine-aided technique.

28. The method of claim 27, wherein the machine-aided technique is a sonography.

29. The method of claim 27, wherein one of the screws in the plurality of screws contains metal and another screw in the plurality of screws operates as a soundwave transmitter.

30. The method of claim 27, wherein the screws contains metal, and wherein the movement of the fasteners is detected with a metal-sensitive machine.
